(19) United States
(12) Patent Application Publication
(10) Pub. No.: US 2025/0325278 A1
(43) Pub. Date: Oct. 23, 2025

(54) Dynamic Bone Compression

(71) Applicant: Arthrex, Inc., Naples, FL (US)

(72) Inventors: Thomas DOONEY, JR., Naples, FL (US); Shaun G. LEBLANC, Naples, FL (US); Jason A. VALENTIN, Fort Myers, FL (US); Alan HIRAHARA, Gold River, CA (US); Thomas M. DEBERARDINO, San Antonio, TX (US)

(73) Assignee: Arthrex, Inc., Naples, FL (US)

(21) Appl. No.: 19/070,814

(22) Filed: Mar. 5, 2025

Related U.S. Application Data
(60) Provisional application No. 63/636,900, filed on Apr. 22, 2024.

(51) Int. Cl. A61B 17/16; A61B 17/04; A61B 17/17

(52) U.S. Cl. CPC A61B 17/1604; A61B 17/0401; A61B 17/1796; A61B 2017/0409

(57) ABSTRACT

Surgical constructs, assemblies and methods of tissue fixation are disclosed. An angled bone punch can form a bone hole (bone opening) and directionally compress around a localized region on the periphery of the bone hole.

### BACKGROUND

The present disclosure relates to the field of surgery and, more particularly, to instruments and methods of surgical repairs.

### SUMMARY

Surgical constructs, assemblies, and kits are disclosed. A bone punch can create a bone hole and purposefully direct and control the compression of the bone. A bone punch can be a cylindrical rod with an angled cut. A gradual taper of the cut pushes the bone in the direction of the flat. A bone punch can create a bone hole or opening while dynamically controlling the direction of compression of the bone about the punch, as the hole is being formed. A bone punch can be employed with a “sled like” open cannula guide, which can be inserted into bone with the direction of the opening facing the desired compression.

Methods of surgeries are also disclosed. In an embodiment, a method of tissue fixation is conducted with a bone punch which has a most distal end with an angled cut to form a bone hole (bone opening) and directionally compress around a localized region on the periphery of the bone hole.

### BRIEF DESCRIPTION OF THE DRAWINGS

FIGS. 1-3 illustrate various views of a distal end of an exemplary bone punch.

FIG. 4 is a top view of a bone hole formed with the bone punch of FIGS. 1-3.

FIG. 5 is a perspective view of a cannula guide for use with the bone punch of FIGS. 1-3.

FIGS. 6 and 7 illustrate steps of an exemplary method of surgery with the bone punch of FIGS. 1-3.

FIGS. 8 and 9 illustrate steps of another exemplary method of surgery including delivering a bone graft with the bone punch of FIGS. 1-3.

FIGS. 10-19 illustrate various views of distal ends of exemplary bone punches.

### DETAILED DESCRIPTION

The present disclosure provides methods, surgical constructs, assemblies, kits and systems for tissue repairs, for example, rotator cuff repair.

A bone punch is provided with an angled or slanted distal face to control the direction of compression of the bone while a bone hole is being formed. This aspect is important for bone fixation devices that can benefit from having denser bone at a certain area of the bone hole where direct forces apply. The bone punch is provided with a body in the shape of a cylindrical rod with a longitudinal axis, a distal end, and a proximal end, wherein the distal end has an angled cut. The angled cut can have a gradual taper. The gradual taper of the cut pushes the bone in the direction of the flat. Depending on the depth of the insertion of the angled punch, some of the hole can be circular and/or flat with compressed bone on one side.

The angled bone punch can be employed with a “sled like” open cannula guide, which can be inserted into the bone first with the direction of the opening facing the desired compression. The “sled” acts as a shield based on its increased surface area over the bone punch. As the angled punch is inserted, the sled helps direct and control the bone compression. The angled bone punch and sled can deliver bone chips (i.e., bone cement, DBM, or similar “bone filler” materials) that can be fed down the sled into the bone hole and then directionally compressed. This aspect is useful when poor weak bone is encountered and needs to be strengthened for device fixation.

Methods of surgery are also disclosed. A method of altering bone density and direct compression around an inner surface of a bone hole includes inter alia the steps of (i) impacting an angled bone punch into bone to form a bone hole having at least two different bone densities around an inner surface of the bone hole. The method can further include: (ii) inserting a sled-like cannula guide into bone; (iii) inserting the angled bone punch through the sled-like cannula guide; and (iv) forming the bone hole with the angled bone punch. The method can further include compressing bone in the bone hole facing an angled cut of the angled bone punch to a first bone density, wherein the first bone density is greater than a density of the bone in the bone hole not facing an angled cut of the angled bone punch.

A method of graft impaction includes the steps of: (i) inserting a sled-like cannula guide into a bone hole; (ii) delivering bone particles or bone-like particles into the bone hole; (iii) inserting an angled bone punch through the sled-like cannula guide and into the bone hole; (iv) directing, with the angled bone punch, the bone particles or bone-like particles into the bone hole into mostly a region of the bone hole; and (v) removing the angled bone punch and the sled-like cannula guide from the bone hole. The method can further include the step of forming the bone hole with the angled bone punch. The method can further include the step of forming the bone hole with another surgical instrument. The method can further include the steps of: inserting a tip of the sled-like cannula guide into the bone hole; inserting the angled bone punch through the sled-like cannula guide in one direction; and locking the angled bone punch relative to the sled- like cannula guide.

A method of graft impaction includes the steps of: (i) impacting a sled-like cannula guide into a bone; (ii) inserting an angled bone punch through the sled-like cannula guide into the bone to form a bone hole; (iii) removing the angled bone punch from the sled-like cannula guide, while the sled-like cannula guide remains into the bone hole; (iv) delivering bone particles or bone-like particles into the bone hole through the sled-like cannula guide; (v) subsequently, inserting the angled bone punch through the sled-like cannula guide and into the bone hole; (vi) directing, with the angled bone punch, the bone particles or bone-like particles into the bone hole into mostly a region of the bone hole; and (vii) removing the angled bone punch and the sled-like cannula guide from the bone hole.

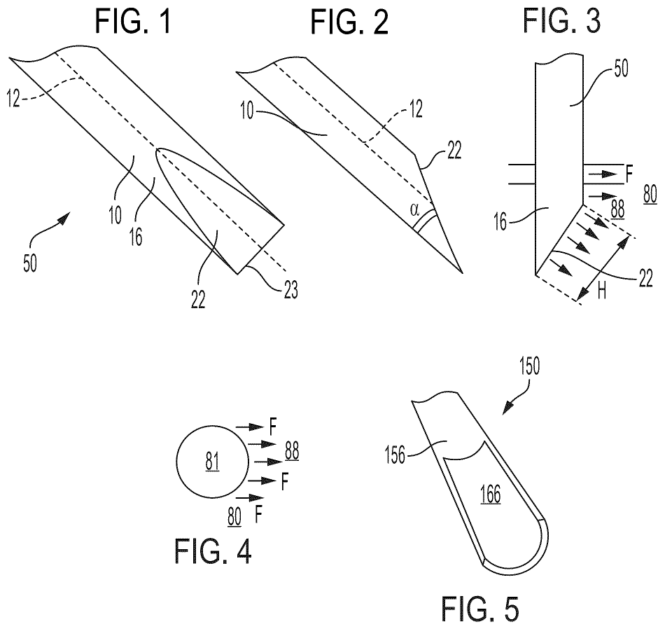

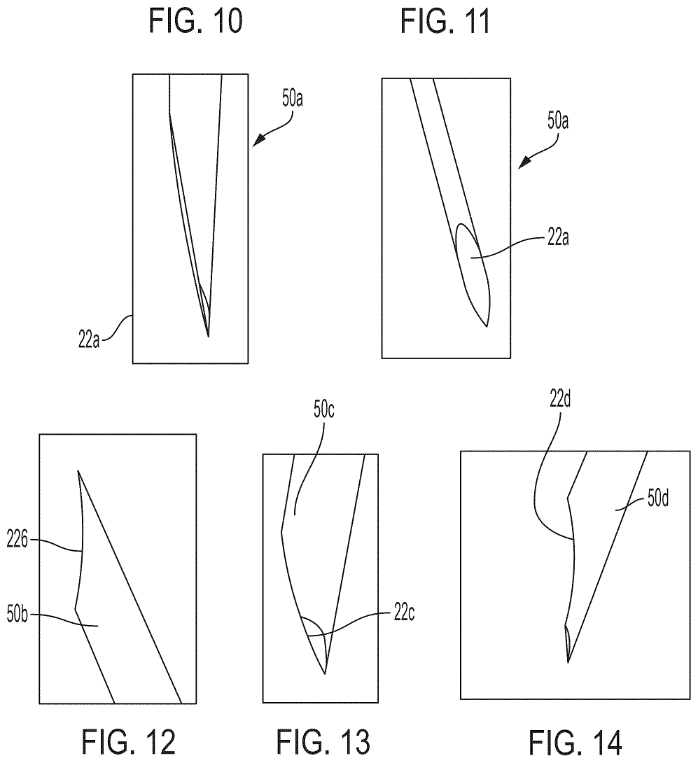

Referring now to the drawings, where like elements are designated by like reference numerals, FIGS. 1-9 illustrate various views of exemplary bone punch 50 and associated cannula guide 150. FIGS. 10-19 illustrate various views of additional exemplary bone punches 50a, 50b, 50c, 50d, 50e, 50f having a distal end (tip) with various configurations.

Bone punch 50 (angled bone punch 50; punch 50; surgical instrument 50) includes a punch body 10 having a general cylindrical shape and a handle (not shown). The punch body 10 is provided with a longitudinal axis 12, a proximal end 14 and a distal end 16. Punch body 10 can be cylindrical (i.e., have a substantially round or circular cross-section) and can be solid or partially solid. In an embodiment, distal end 16 is solid.

An angle cut “α” of less than sixty degrees and in many implementations less than forty-five degrees relative to the longitudinal axis 12 is provided at the distal end 16, as shown in FIGS. 1 and 2. The angled cut provides the bone punch 50 with at least one angled face 22. In an embodiment, angled face 22 is substantially planar and forms and angle of less than about forty-five degrees, preferably about ten to less than about forty-five degrees. Angled face 22 is tapered at an angle of about ten to less than about sixty degrees (in many implementations about ten to forty-five degrees) and can be machined into the distal end 16 until the face 22 meets most distal end 23 of body 10. In other embodiments, angled face 22 is partially flat and forms an angle of less than forty-five degrees. In other embodiments, angled face 22 is flat and forms an angle of less than forty-five degrees. In other embodiments, angled face 22 is either convex or concave and forms an angle of less than forty-five degrees.

In yet additional embodiments, and as detailed below with reference to FIGS. 10-19, angled face 22 can have multiple combined configurations such as combination of concave, convex and/or flat; combination of two or more flat surfaces with different angulations (i.e., a first flat surface at a first angle adjacent and extending to a second flat surface at a second angle, wherein the first angle is different from the second angle; or a first flat surface at a first angle circumferentially adjacent at least another flat surface at a second angle, wherein the first angle is different from the second angle); general unidirectional orientation; or general multidirectional orientation, among others.

Bone punch 50 can be manufactured from metals, metal alloys, non-metals, plastics, or combinations thereof. Bone punch 50 can be a metal instrument produced by 3D printing, injection molding, or machining. Bone punch 50 can be formed of stainless steel; can be disposable; can be reusable. Bone punch 50 is a simple construct that allows precise, accurate and convenient control of bone compression within a bone hole, as well as control of direction of graft impaction within a bone hole.

FIGS. 3 and 4 schematically illustrate angled bone punch 50 impacted and/or inserted into bone 80. Force “F” created with the angled bone punch 50 direct the bone particles (of bone 80) into bone hole 81 into mostly a single region 88 of the bone hole 81. As shown in FIG. 4, for example, the angled or slanted distal face 22 of the bone punch 50 controls the direction of compression of the bone 80 while bone hole 81 is being formed. In this manner, a denser bone is formed at a certain area (such as region 88) of the bone hole 81 where direct forces F apply. The gradual taper of the cut pushes the bone in the direction of the flat 22. If the punch 50 is inserted past the proximal end of the flat 22, depending on the depth of the insertion of the angled punch, some of the hole 81 can be circular and/or flat with compressed bone area 88 on one side.

The slanted face 22 of the angled bone punch 50 allows formation of a prepared non-uniform bone hole. The angled bone punch 50 expands out bone 80 in a non-radial or non-circumferential fashion; the bone 80 is non-uniformly expanded and non-uniformly compressed (i.e., dynamically compressed) around the periphery of bone hole 81. Bone fixation devices greatly benefit from having denser bone at certain areas of the bone hole (such as region 88) where direct forces apply to compress the bone (to push the bone in the direction of the flat).

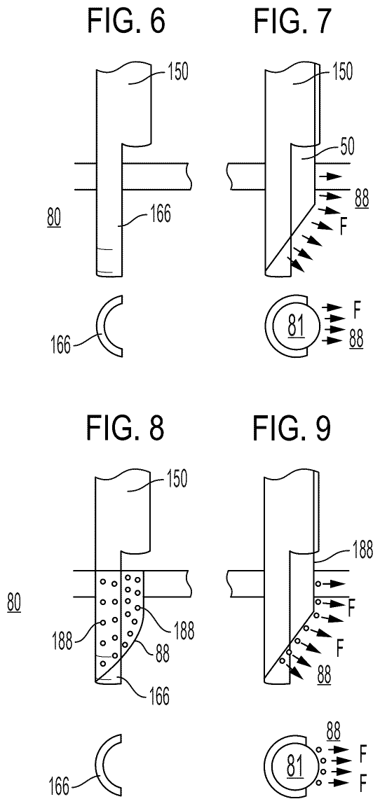

Reference is now made to FIG. 5 which illustrates cannula guide 150 (“sled-like” cannula guide 150; guide 150) that can be used by itself or in combination with a bone punch such as angled bone punch 50. Cannula guide 150 is provided with a cannulated cylindrical body having a distal end 156 with an opening 166 having about a semicircular configuration. Preferably, and as shown in FIG. 6, the cut 166 in the sled is less than a semicircle. The sled-like open cannula guide 150 can be inserted into the bone first with the direction of the opening facing the desired bone compression. The sled-like open cannula guide 150 can be manufactured from metals, metal alloys, non-metals, plastics, or combinations thereof. The sled-like open cannula guide 150 can be a metal guide produced by 3D printing, injection molding, or machining. The sled-like open cannula guide 150 can be formed of stainless steel; can be disposable; can be reusable. The cannula is a simple construct that allows precise, accurate and convenient delivery of bone particles (or similar graft particles) as well as control of the direction of bone compression into a bone hole. In one configuration, sled-like open cannula guide 150 can be manufactured from a hollow cylindrical tube, tubing, pipe or similar element having a most distal portion of the wall cut away (in a direction about parallel to the longitudinal axis of the tube) to allow formation of a semi-circle portion in the remaining wall, or a portion with a curvature of a semi-circle or less than a semi-circle.

Bone punch 50 and/or cannula guide 150 can be employed in various methods of surgery that can include, for example, bone hole formation for tissue repairs and fixation, bone compression, graft impaction, etc. In an embodiment, angled bone punch 50 can be impacted into bone 80 to form a bone hole 81 having at least two different bone densities around an inner surface of the bone hole 81. Bone region 88 (a first bone region) has a first bone density which is greater than a second bone density of another region (a second bone region) of bone hole 81.

FIGS. 6-9 illustrate exemplary methods of altering bone density and compression around an inner surface of a bone hole. FIGS. 6 and 7 illustrate first the insertion of the sled-like cannula guide 150 into bone 80 followed by sliding or inserting the angled bone punch 50 through the sled-like cannula guide 150 to form the bone hole 81 with the angled bone punch 50. The method can further include compressing bone 80 in the bone hole 81 facing an angled cut 22 of the angled bone punch 50 to a first bone density, wherein the first bone density is greater than a density of the bone in the bone hole 81 not facing an angled cut 22 of the angled bone punch 50.

FIGS. 8 and 9 illustrate an exemplary method of graft impaction. Sled-like cannula guide 150 is inserted into a bone hole 80. Bone particles/chips/bone-like particles 188 are delivered through the cannula guide 150 into the bone hole 80. An angled bone punch (such as angled bone punch 50 or a different bone punch) can be inserted through the sled-like cannula guide 150 and into the bone hole 81, as shown in FIG. 9. With the angled bone punch 50, bone particles 188 (bone chips or bone-like particles 188) are directed into the bone hole 81 into mostly a region 88 of the bone hole while the bone 80 is also compressed in the same region 88. Bone hole 81 can be formed with the angled bone punch 50 before the step of inserting the cannula guide 150 into the bone hole. Alternatively, bone hole 81 can be first formed with another surgical instrument (e.g., another bone punch) and then the sled-like cannula guide 150 can be introduced.

Bone particles 188 can include bone chips; bone cement; DBM; mineralized cortical allograft particles or particulates; mineralized cortical bone particles; mineralized cancellous bone particles; bioresorbable and non-resorbable bone substitutes such as hydroxyapatite (HAP) and beta-tricalcium (ß-TCP) particles; synthetic mineral bone graft granules; and/or other bone void fillers known in the art. If desired, additional biologics, for example, bone-to-bone biologics can be employed with the bone particles 188. Various medicinal and/or therapeutic agents, for example, antiseptics, antibiotics, drugs, pharmaceutical agents, hormones, and growth materials (for example, autogenous growth factors such as platelet-rich plasma (PRP), or autologous factors) among many others can be added at the hole site to aid in the wound closure and overall graft healing.

The angled bone punch 50 can be introduced through the sled-like cannula guide 150 in one direction. The angled bone punch 50 can be locked relative to the sled-like cannula guide 150, to prevent movement and allow increased stability of the overall procedure. Locking can be conducted, for example, by introducing the bone punch 50 through the cannula guide in one direction and securing a little knob (insert or protuberance) of the bone punch into a matching groove of the cannula guide 150. Together with the bone punch 50, the sled-like open cannula guide 150 controls both the delivery and direction of the bone compression.

FIGS. 10-19 illustrate additional exemplary bone punches 50a, 50b, 50c, 50d, 50e, 50f which are similar to angled bone punch 50 described above in that they have characteristics similar to the bone punch 50 and they can also be employed for the bone density augmentation procedures detailed above, with or without the sled-like cannula guide 150. Bone punches 50a, 50b, 50c, 50d, 50e, 50f differ, however, from bone punch 50 in the design of the distal end 16 and angled face 22. For example, FIGS. 10-14 illustrate bone punches 50a, 50b, 50c which allow for unidirectional bone density augmentation. Face 22a of bone punch 50a is angled and flat and terminates into a sharp point/tip, while face 22b of bone punch 50b is concave and face 22c of bone punch 50c is convex. Face 22d of bone punch 50d is multifaceted, i.e., it includes a concave, convex, or alt angle which ends distally into a flat surface.

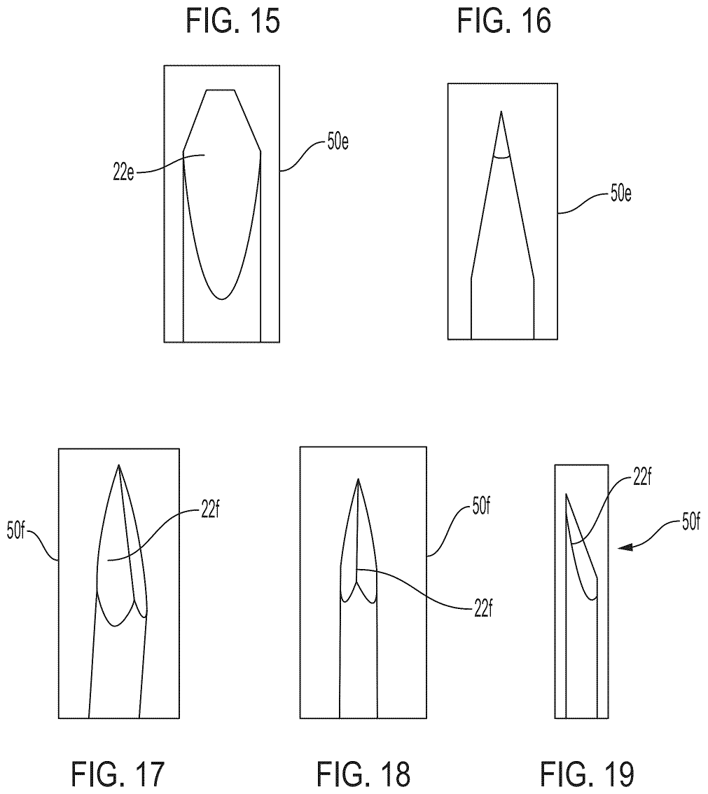

FIGS. 15-19 illustrate bone punches 50e and 50f which allow for multidirectional bone density augmentation. Bone punch 50e has a bisecting angled configuration. Bone punch 50f is multifaceted and multi-angular, i.e., it can include concave, convex, or flat faces/facets at same or different angles relative to the longitudinal axis which converge into a single tip/point (as shown in FIGS. 17-19) or into a line. In additional embodiments, a bone punch can be machined with a slanted face 22 having two different angles, for example, a first angle and a second angle, wherein the first angle is different from the second angle, and wherein both the first and second angles are less than about 45 degrees. In other embodiments, a bone punch can be machined with multiple slanted faces extending circumferentially around the distal end, wherein at least one of the multiple slanted faces has an angle different from the angles of the remaining multiple slanted faces. The multiple slanted faces can all converge into a single point (most distal tip) or into a single line (most distal line). In additional embodiments, some of the multiple slanted faces can converge into a first line or first point, whereas other of the multiple slanted faces can converge into a second line or second point. Preferably, the angles are less than about 45 degrees. The multiple slanted faces can have a planar (flat configuration) and/or a non-planar (non-flat configuration), or combinations thereof.

Bone punch 50, 50a, 50b, 50c, 50d, 50e, 50f and sled-like open cannula guide 150 have applicability to various open and arthroscopic procedures, with particular application to formation of bone holes with different bone densities for fixation of various fixation devices such as screws, anchors, implants, bone plates, etc. Bone punch 50, 50a, 50b, 50c, 50d, 50e, 50f and sled-like open cannula guide 150 can be employed in procedures for re-approximating bone to bone or soft tissue to bone, for example, shoulder rotator cuff repairs, capsulolabral reconstruction, SLAP repairs, as well as ankle, knee, elbow or foot repairs.

Bone punch 50, 50a, 50b, 50c, 50d, 50e, 50f and sled-like open cannula guide 150 described above can be included in a surgical kit, assembly, or system to simplify the surgeon's task of selecting a specific instrument and to aid in the overall surgical procedure. A surgical kit for an arthroscopic surgical repair can include one or more bone punch 50, 50a, 50b, 50c, 50d, 50e, 50f and one or more sled-like open cannula guide 150. The surgical kit can include awls or equivalent devices, as well as drills or additional bone-penetrating devices. The surgical kit can also include a tensioner and/or bone plates and associated instrumentation. The surgical kit can include fixation devices (such as anchors, screws, suture-button constructs, plates, etc.) and flexible members (such as sutures) to be employed in conjunction with the bone tunnels, bores or holes formed by and with bone punch 50, 50a, 50b, 50c, 50d, 50e, 50f and sled-like open cannula guide 150.

In an exemplary embodiment, a fixation device employed with bone punch 50, 50a, 50b, 50c, 50d, 50e, 50f and/or cannula guide 150 can be an all-suture soft anchor (soft suture anchor) provided with a soft anchor sleeve (sheath, tubular member) with two open ends and one or more flexible shuttling strands extending through the soft anchor sleeve (sheath). The at least two flexible strands may extend through the sleeve in similar or different directions and/or orientations and/or locations. The flexible strands can include high strength suture such as FiberWire® suture, TigerWire® suture, FiberTape® suture tape, among many others. The flexible sleeve with the one or more shuttling strands is secured into or onto bone, and the strands allow passing of additional flexible strands such as tapes to pass over soft tissue and be secured into bone to approximate soft tissue to bone. Details of an exemplary soft suture anchor with a soft anchor sleeve (sheath or tubular member) and flexible shuttling strands are set forth, for example, in US application Ser. No. 15/998,516 entitled “Methods of Tissue Repairs” filed on Aug. 16, 2018, the disclosure of which is incorporated by reference in its entirety herein.

A bone punch 50, 50a, 50b, 50c, 50d, 50e, 50f includes a body 10 with a distal end 12 provided with at least one angled or slanted distal face 22 to control the direction of compression of the bone 80 while a bone hole 81 is being formed. This aspect is important for bone fixation devices that can benefit from having denser bone at a certain area 88 of the bone hole 81 where direct forces F would apply. The angled cut can have a gradual taper. The gradual taper of the cut pushes the bone 80 in the direction of the flat 22. Depending on the depth of the insertion of the angled punch, some of the hole 81 can be circular and/or flat with compressed bone on one side 88.

The angled bone punch 50, 50a, 50b, 50c, 50d, 50e, 50f can be employed with a “sled like” open cannula guide 150, which can be inserted into the bone 80 first with the direction of the opening 166 facing the desired compression. The “sled” acts as a shield based on its increased surface area over the bone punch 50, 50a, 50b, 50c, 50d, 50e, 50f. As the angled punch 50, 50a, 50b, 50c, 50d, 50e, 50f is inserted, the sled 150 helps direct and control the bone compression in region 88 of the bone hole 81 of bone 80. The angled bone punch 50, 50a, 50b, 50c, 50d, 50e, 50f and sled 150 can help deliver bone particles 188 (e.g., bone cement, bone chips, DBM, or similar “bone filler” materials) that can be fed down the sled 150 into the bone hole 81 and then directionally compressed. This aspect is useful when poor weak bone is encountered and needs to be strengthened for device fixation.

Methods of surgery are also disclosed. A method of altering the bone density and controlling the compression around an inner surface of a bone hole 81 includes inter alia the steps of (i) impacting an angled bone punch 50, 50a, 50b, 50c, 50d, 50e, 50f into bone 80 to form a bone hole 81 having at least two different bone densities around an inner surface of the bone hole 81. The method can further include: (ii) inserting a sled-like cannula guide 150 into bone 80; (iii) inserting the angled bone punch 50, 50a, 50b, 50c, 50d, 50e, 50f through the sled-like cannula guide 150; and (iv) forming the bone hole 81 with the angled bone punch 50, 50a, 50b, 50c, 50d, 50e, 50f. The method can further include compressing bone 80 in the bone hole 81 facing an angled cut 22 of the angled bone punch 50, 50a, 50b, 50c, 50d, 50e, 50f to a first bone density of region 88, wherein the first bone density is greater than a density of the bone in the bone hole not facing an angled cut of the angled bone punch.

A method of graft impaction includes the steps of: (i) inserting a sled-like cannula guide 150 into a bone hole 81; (ii) delivering bone particles or bone-like particles 188 into the bone hole 81 and through the sled-like cannula guide 150; (iii) inserting an angled bone punch 50, 50a, 50b, 50c, 50d, 50e, 50f through the sled-like cannula guide 150 and into the bone hole 81; (iv) directing, with the angled bone punch 50, 50a, 50b, 50c, 50d, 50e, 50f and the sled-like cannula guide 150, the bone particles or bone-like particles 188 into the bone hole 81 into mostly a region 88 of the bone 80 and adjacent bone hole 81; and (v) removing the angled bone punch 50, 50a, 50b, 50c, 50d, 50e, 50f and the sled-like cannula guide 150 from the bone hole 81. The method can further include the step of forming the bone hole 81 with the angled bone punch 50, 50a, 50b, 50c, 50d, 50e, 50f. The method can further include the step of forming the bone hole 81 with a surgical instrument different from the angled bone punch 50, 50a, 50b, 50c, 50d, 50e, 50f. The method can further include the steps of: inserting a tip of the sled-like cannula guide 150 into the bone hole 81; sliding the angled bone punch 50, 50a, 50b, 50c, 50d, 50c, 50f through the sled-like cannula guide 150 in one direction; and locking the angled bone punch 50, 50a, 50b, 50c, 50d, 50e, 50f relative to the sled-like cannula guide 150.

A method of graft impaction includes the steps of: (i) impacting a sled-like cannula guide 150 into a bone 80; (ii) inserting an angled bone punch 50, 50a, 50b, 50c, 50d, 50e, 50f through the sled-like cannula guide 150 into the bone 80 to form a bone hole 81; (iii) removing the angled bone punch 50, 50a, 50b, 50c, 50d, 50e, 50f from the sled-like cannula guide 150, while the sled-like cannula guide remains into the bone hole 81; (iv) delivering bone particles 188 or bone-like particles 188 into the bone hole 81 through the sled-like cannula guide 150; (v) subsequently, inserting the angled bone punch 50, 50a, 50b, 50c, 50d, 50e, 50f through the sled-like cannula guide 150 and into the bone hole 81; (vi) directing, with the angled bone punch 50, 50a, 50b, 50c, 50d, 50e, 50f, the bone particles 188 or bone-like particles 188 into the bone hole into mostly a region 88 of the bone hole 81; and (vii) removing the angled bone punch 50, 50a, 50b, 50c, 50d, 50e, 50f and the sled-like cannula guide 150 from the bone hole 81.

The term “sled-like cannula guide” is defined as a tube, tubing, or hollow pipe manufactured in round or oval cross-sections with a varying or same diameter, and with part of the wall at the most distal end of the tube being cut in a direction about parallel to the longitudinal axis of the tube, to create an opening in the wall and to allow the remaining wall to have an about semicircular configuration or less than about semicircular configuration.

The term “high strength suture” is defined as any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body.

What is claimed is:

1. An angled bone punch for dynamic bone compression, comprising:
a shaft having a longitudinal axis, a proximal end, and a distal end; and
at least one angled distal face located at the distal end, wherein each of the plurality of angled distal faces forms an angle of less than about 60 degrees with the longitudinal axis, and wherein the at least one angled distal face terminates in a most distal line at a most distal end of the shaft.

2. The angled bone punch of claim 1, wherein the at least one angled distal face includes a convex surface, a concave surface, a planar surface, or a combination thereof.

3. The angled bone punch of claim 1, wherein the at least one angled distal face directs compression of bone in a bone hole in a region of the bone facing the at least one angled distal face.

4. The angled bone punch of claim 3, wherein the at least one angled distal face engages the region of the bone hole and compacts the bone.

5. The angled bone punch of claim 4, wherein the region of the bone has a first bone density, and wherein another region of the bone not facing the at least one angled distal face has a second bone density.

6. The angled bone punch of claim 5, wherein the first bone density is different from the second bone density.

7. The angled bone punch of claim 5, wherein the first bone density is greater than the second bone density.

8. An angled bone punch for dynamic bone compression, comprising:
a shaft having a longitudinal axis, a proximal end, and a distal end; and
at least one angled distal face located at the distal end, wherein at least one angled distal face forms an angle of less than about 60 degrees with the longitudinal axis, and wherein the at least one angled distal face converges into a most distal point at a most distal end of the shaft.

9. The angled bone punch of claim 8, wherein the at least one angled distal face consists of two planar surfaces converging into the most distal point.

10. The angled bone punch of claim 8, wherein the at least one angled distal face consists of a planar surface and a convex surface converging into the most distal point.

11. The angled bone punch of claim 8, wherein the at least one angled distal face consists of a concave or convex surface terminating into a flat surface at the most distal point.

12. The angled bone punch of claim 8, wherein the at least one angled distal face directs compression of bone in a bone hole in a region of the bone facing the at least one angled distal face.

13. A surgical kit for dynamic bone compression, comprising:
an angled bone punch having a shaft with a longitudinal axis, a proximal end and a distal end; and at least one angled distal surface located at the distal end, wherein the at least one angled distal surface forms an angle of less than about 60 degrees with the longitudinal axis of the shaft; and
an open cannula guide having a “sled-like” configuration.

14. The surgical kit of claim 13, wherein the open cannula guide is configured to be inserted into a bone hole to assist in delivery and direction of bone material into the bone hole.

15. The surgical kit of claim 14, wherein the bone material is bone chips, bone particles, or DMB.

16. The surgical kit of claim 13, wherein at least one of the angled bone punch and the open cannula guide is disposable.

17. The surgical kit of claim 13, further comprising an obturator.

18. The surgical kit of claim 13, further comprising at least one cutting instrument.

19. The surgical kit of claim 13, further comprising at least one fixation device.

20. The surgical kit of claim 19, wherein the at least one fixation device is a soft suture anchor comprising a flexible tubular sleeve or sheath and a plurality of flexible strands extending through a passage of the flexible tubular sleeve or sheath.
